(19) United States
(12) Patent Application Publication
(10) Pub. No.: US 2005/0202107 A1
(43) Pub. Date: Sep. 15, 2005

(54) Use of ginkgo biloba extracts in order to promote muscle mass to the detriment of fatty mass

(76) Inventor: Yves Christen, PARIS (FR)

Correspondence Address: HEDMAN & COSTIGAN P.C., 1185 AVENUE OF THE AMERICAS, NEW YORK, NY, 10036, US

(21) Appl. No.: 10/517,219

(22) PCT Filed: Jul. 15, 2003
(86) PCT No.: PCT/FR03/02230

§ 371 Date: Dec. 3, 2004

(30) Foreign Application Priority Data
Jul. 16, 2002 (FR) 02/08941

(51) Int. Cl. A61K 31/365; A61K 35/78

(52) U.S. Cl. 424/752; 514/468

(57) ABSTRACT

The invention relates to the use of Ginkgo biloba extracts and, in particular, Ginkgo biloba extracts comprising between 20 and 30% flavoneglycosides, between 2.5 and 4.5% in total of ginkgolides A, B, C and J, between 2 and 4% bilobalide, less than 10% proanthocyanidins and less than 10 ppm alkylphenol-type compounds, in order to prepare a medicament that is intended to promote muscle mass to the detriment of fatty mass in patients looking to loose or gain weight by means of a diet and/or medical treatment.

The present patent application relates to the use of Ginkgo biloba extracts for the preparation of a medicament intended to promote muscle mass to the detriment of fatty mass.

Obesity is a real public health problem. Certain medicaments do allow stimulation of a weight loss or gain, but the treatment generally weakens the patients in whom the weight loss or gain occurs often to the detriment of the ratio of muscle mass to fatty mass.

The Applicant has now discovered that the administration of Ginkgo biloba extracts to subjects trying to lose or gain weight has the beneficial effect of promoting muscle mass to the detriment of fatty mass. Moreover, the Applicant was able to observe that said administration stimulates a weight loss in the overweight subject to whom it is administered.

Generally, the invention relates to the use of Ginkgo biloba extracts for the preparation of a medicament intended to treat weight problems, both in people seeking to gain weight and to lose it.

The invention relates in particular to the use of Ginkgo biloba extracts for the preparation of a medicament intended for weight loss. According to the invention, the weight loss in the subject treated is at least 4 or 5%, and more preferably at least 6, 8 or 10% of their total body weight.

According to a particular variant of the invention, the Ginkgo biloba extracts are used for the preparation of a medicament intended to promote muscle mass to the detriment of fatty mass in patients seeking to lose or gain weight. These patients are optionally subjected to a diet and/or to another medical treatment.

In other words, when, according to the invention, the patient seeking to gain or lose weight is treated with a Ginkgo biloba extract concomitant with their diet or other medical treatment, the ratio R equal to their muscle mass Mₘ divided by their total body weight Mₜ tends to remain stable or, in most cases, to increase. Preferably, the increase thus obtained after a period of treatment of at least one month with an extract of Ginkgo biloba will be greater than or equal to 5%, and more preferably greater than or equal to 6 or 8 or 10%.

The Ginkgo biloba extracts which can be used according to the invention are such that they comprise at least flavoneglycosides and/or a ginkgolide or ginkgolides. Preferably, the flavoneglycosides and/or the ginkgolide or ginkgolides are present at least at a level of 25% by weight, more preferably at least at a level of 30% by weight and still more preferably at least at a level of 50% by weight in the Ginkgo biloba extract used for the preparation of the medicament according to the invention. Moreover, the proportion of compounds of alkylphenol type in the Ginkgo biloba extract used according to the invention is preferably less than 10 ppm, more preferably less than 5 ppm and still more preferably less than 1 ppm. If appropriate, the ginkgolide or ginkgolides can be replaced with their acetylated homologues, their alkoxylated homologues or their glycosylated homologues (such as for example the compounds of general formula (I) described below).

Preferably, the Ginkgo biloba extract used for the preparation of a medicament according to the invention is enriched with flavoneglycosides and/or ginkgolides. It can for example be an EGb 761® type extract. According to another variant of the invention, the Ginkgo biloba extract used for the preparation of a medicament according to the invention is any Ginkgo biloba extract containing flavoneglycosides, ginkgolides and bilobalide, for example a CP 401 type extract.

By EGb 761® type extract, is meant an extract with a composition more or less identical to that of the standardized EGb 761® extract as it has been defined in particular in the following article: K. Drieu, La presse médicale, 31, 25 Sep. 1986, supplement dedicated to the Ginkgo biloba extract (EGb 761®), 1455-1457; or in the European patents EP 431 535 and EP 431 536; by EGb 761® type extract, is therefore meant in particular the Ginkgo biloba extracts comprising from 20 to 30% of flavoneglycosides, from 2.5 to 4.5% in total of ginkgolides A, B, C and J, from 2 to 4% of bilobalide, less than 10% of proanthocyanidines and less than 10 ppm (preferably less than 5 ppm and still more preferably less than 1 ppm) of compounds of alkylphenol type, preferably the Ginkgo biloba extracts comprising from 22 to 36% of flavoneglycosides, from 2.5 to 3.5% in total of ginkgolides A, B, C and J, from 2.5 to 3.5% of bilobalide, less than 8% of proanthocyanidines and less than 10 ppm (preferably less than 5 ppm and still more preferably less than 1 ppm) of compounds of alkylphenol type, and in particular the Ginkgo biloba extracts comprising approximately 24% of flavoneglycosides, 3.1% in total of ginkgolides A, B, C and J, 2.9% of bilobalide, 6.5% of proanthocyanidines and less than 1 ppm of compounds of alkylphenol type.

By CP 401 type extract, is meant extracts such as those which are presented in the U.S. Pat. No. 5,389,370, in particular the Ginkgo biloba extracts comprising from 5.5 to 8% in total of ginkgolides A, B, C and J, from 40 to 60% of flavoneglycosides and from 5 to 7% of bilobalide, preferably the Ginkgo biloba extracts comprising from 6.5 to 7.5% in total of ginkgolides A, B, C and J, from 45 to 55% of flavoneglycosides and from 5.5 to 6.5% of bilobalide and quite particularly the extracts comprising approximately 7 % in total of ginkgolides A, B, C and J, 50% of flavoneglycosides and 6% of bilobalide.

By extension, the EGb 761® or CP 401 type extracts, the ginkgolides of which have been replaced with their homologues of general formula (I) described later, are also in the same category as the EGb 761® or CP 401 type extracts.

According to one variant of the invention, at least part of the ginkgolide or ginkgolides can be replaced with the compounds of general formula (I) 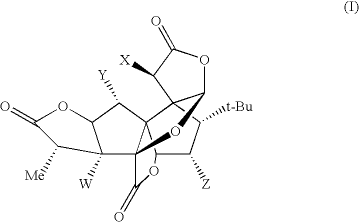
in which W, X, Y and Z independently represent the H, OH, linear or branched alkoxy or O-G_{S} radicals, G_{S}-OH representing a mono- or a disaccharide, or one of their derivatives or analogues,

it being understood that at least one of W, X, Y or Z represents an O-G_{S} radical.

Preferably, the compounds of general formula (I) described previously are such that X represents an OH or O-G_{S} radical, G_{S}-OH representing a mono- or a disaccharide, or one of their derivatives or analogues, and:
either W represents an OH or O-G_{S} radical, Y represents H and Z represents H;
or W represents an OH or O-G_{S} radical, Y represents an OH or O-G_{S} radical and Z represents H;
or W represents an OH or O-G_{S} radical, Y represents an OH or O-G_{S} radical and Z represents an OH or O-G_{S} radical;
or W represents an OH or O-G_{S} radical, Y represents H and Z represents an OH or O-G_{S} radical;
or W represents H, Y represents an OH or O-G_{S} radical and Z represents an OH or O-G_{S} radical;
or W represents an OH or O-G_{S} radical, Y represents a linear or branched alkoxy radical and Z represents H.

For the preparation of the compounds of general formula (I), a person skilled in the art can refer to the PCT Patent Application WO 98/52959 or to the U.S. Pat. No. 6.143.725.

According to one aspect of the invention, the patient has weight to lose. In this case, the Ginkgo biloba extract can for example be administered to the patient in combination with a medicament containing orlistat (for example Xenical®), sibutramine or one of its pharmaceutically acceptable salts (for example Sibutral®), hydroalcoholic extracts of green tea (for example Exolise® or Mincifit®) or from green tea (for example Camiline®), or with any other medicament intended to stimulate weight loss. Preferably according to the invention, the Ginkgo biloba extract is administered in combination with a medicament containing orlistat (for example Xenical®), sibutramine or one of its pharmaceutically acceptable salts (for example Sibutral®). However, when the weight to be lost is relatively small (for example less than or equal to 5%, or less than or equal to 10% of total body weight), the patient can simply receive the Ginkgo biloba extract in addition to the diet which he has been prescribed.

The invention therefore also relates to a product comprising at least one Ginkgo biloba extract as described previously in combination with at least one compound chosen from orlistat, sibutramine or one of its pharmaceutically acceptable salts, hydroalcoholic extracts of green tea or from green tea for a use which is simultaneous, separate or spread over time in the treatment of excess weight.

By simultaneous therapeutic use, is meant in the present application an administration of several active ingredients by the same route and at the same time. By separate use, is meant in particular in the present application an administration of several active ingredients at approximately the same time by different routes. By therapeutic use spread over time, is meant in the present application an administration of several active ingredients at different times and in particular an administration method according to which all the administration of one of the active ingredients is carried out before the administration of the other or others begins. Thus one of the active ingredients can be administered for several months before administering the other active ingredient or active ingredients. There is no simultaneous treatment in this case.

According to the other aspect of the invention, the patient has weight to gain. In this case, the Ginkgo biloba extract can be administered to the patient in combination with a medicament such as fenugreek (for example Fénugrène®) or any other medicament intended to stimulate weight gain. However, when the weight to be gained is relatively small (for example less than or equal to 5%, or less than or equal to 10% of the total body weight), the patient can simply receive Ginkgo biloba extract in addition to the diet which he has been prescribed.

The invention therefore also relates to a product comprising at least one Ginkgo biloba extract as described previously in combination with fenugreek for a use which is simultaneous, separate or spread over time in the treatment of a weight deficiency.

According to the invention, the patient who has weight to lose or to gain can be an animal or a human. Among the animals for which the invention can be used, in particular there may be mentioned dogs, cats, bovines, ovines, poultry (chickens, turkeys, ducks, etc.) or horses. Preferably, the patient is a human.

The pharmaceutical compositions comprising a Ginkgo biloba extract can be in the form of solids, for example powders, granules, tablets, gelatin capsules, liposomes, suppositories or patches. Appropriate solid supports can be, for example, calcium phosphate, magnesium stearate, talc, sugars, lactose, dextrin, starch, gelatin, cellulose, methyl cellulose, sodium carboxymethyl cellulose, polyvinylpyrrolidine and wax.

The pharmaceutical compositions comprising a Ginkgo biloba extract can also be in liquid form, for example, solutions, emulsions, suspensions or syrups. Appropriate liquid supports can be, for example, water, organic solvents such as glycerol or glycols, as well as their mixtures, in varying proportions, in water.

The administration of a medicament according to the invention can be carried out by topical, oral, parenteral route, by injection (intramuscular, sub-cutaneous, intravenous, etc.), etc.

The daily dosage of Ginkgo biloba extract envisaged is comprised between 0.1 mg to 10 g according to the concentration of active ingredient of the extract and the seriousness of the weight problems of the subject to be treated. The final decision will be made by the attending doctor or veterinarian.

The term “approximately” refers to an interval around the value considered. As used in the present application, “approximately X” signifies an interval of X minus 10% of X to X plus 10% of X, and preferably an interval of X minus 5% of X to X plus 5% of X.

Unless it is specified otherwise, all the technical and scientific terms used here have the same meaning as that usually understood by a specialist in the field to which this invention belongs. Similarly, all the publications, patent applications, all the patents and all other references mentioned here are incorporated by way of reference.

In order to show the benefit of the use of Ginkgo biloba extracts as described previously in the treatment of weight problems, the test described hereafter can be carried out. Other tests which aim for example to determine the body composition and in particular the ratio of fatty mass to non-fatty mass (cf. Chumlea et al., Nutrition, Health & Aging, 1(1), 7-12) can also be carried out in order to achieve the same result.

Pharmacological Section

Comparitive Measurement of the Development of Body Weight in Aged Rats Treated or not Treated with EGb 761:

The aged laboratory rats have, due to their captivity, a natural tendency to gain weight. Two groups of aged (22 months) Wistar rats are created, one being constituted by 11 rats which receive normal drinking water for 5 weeks and the other being constituted by 12 rats which receive drinking water containing 75 mg per kg of standardized Ginkgo biloba extract EGb 761®. The rats are weighed before the start and after the end of the treatment.

Results Obtained:

i) The measurements of the weight of the rats produce the following results:

| | Non-treated aged | Treated |
|---|---|---|
| | rats | aged rats |
| Average weight at 22 months (g) | 605.64 | 594.10 |
| Average weight at 22 months and 5 | 619.33 | 570.11 |
| weeks (g) | | |

In other words, in the space of a few weeks, the non-treated rats have gained 2.2% in body weight while the treated rats have lost 4% of their body weight.

ii) Moreover, it is observed above all that the ratio of muscle weight in relation to the total body weight is 0.53 in the treated rats compared to 0.42 for the non-treated rats.

Consequently, it is seen that a treatment with the standardized Ginkgo biloba extract EGb 761® does allow, on the one hand, weight loss in the rats which have a tendency to become obese, and, on the other hand, a promotion of their muscle mass to the detriment of their fatty mass.

1. (canceled)

2. The method of claim 11, wherein the flavoneglycosides and/or the ginkgolide or ginkgolides are present at least at a level of 25% by weight in the Ginkgo biloba extract.

3. The method of claim 11, wherein the Ginkgo biloba extract comprises from 20 to 30% of flavoneglycosides, from 2.5 to 4.5% in total of ginkgolides A, B, C and J, from 2 to 4% of bilobalide, less than 10% of proanthocyanidines and less than 10 ppm of compounds of alkylphenol type.

4. The method of claim 3, wherein at least one part of the ginkgolides A, B, C and J is replaced with the compounds of the formula 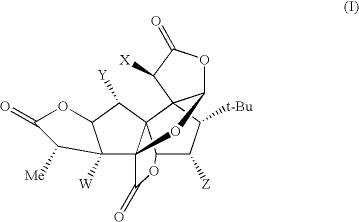
wherein W, X, Y and Z are independently selected from the group consisting of H, OH, alkoxy and —O-G_{S} radicals, G_{S}-OH being a mono- or a disaccharide, or one of their derivatives or analogues,
it being understood that at least one of W, X, Y or Z is —O-G_{S}.

5. The method of claim 4, wherein the compounds of formula (I) are such that X is —OH or —O-G_{S}, G_{S}-OH being a mono- or a disaccharide, or one of their derivatives or analogues, and:
either W is —OH or —O-G_{S}, Y is H and Z is H;
or W is —OH or —O-G_{S}, Y is —OH or —O-G_{S} and Z is H;
or W is —OH or —O-G_{S}, Y is —OH or —O-G_{S} and Z is —OH or —O-G_{S};
or W is —OH or —O-G_{S}, Y is H and Z is —OH or —O-G_{S};
or W is H, Y is —OH or —O-G_{S} and Z is —OH or —O-G_{S};
or W is —OH or —O-G_{S}, Y is alkoxy and Z is H.

6. The method of claim 11, wherein the Ginkgo biloba extract comprises from 5.5 to 8% in total of ginkgolides A, B, C and J, from 40 to 60% of flavoneglycosides and from 5 to 7% of bilobalide.

7. The method of claim 6, wherein a part at least of the ginkgolides A, B, C and J is replaced with the compounds of the formula 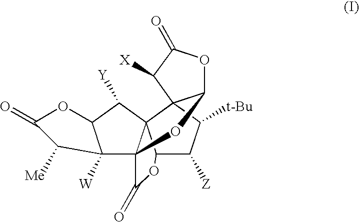
wherein W, X, Y and Z are independently selected from the group consisting of H, OH, alkoxy and —O-G_{S}, G_{S}-OH being a mono- or a disaccharide, or one of their derivatives or analogues,
it being understood that at least one of W, X, Y or Z is —O-G_{S}.

8. The method of claim 7, wherein the compounds of formula (I) are such that X is —OH or —O-G_{S}, G_{S}-OH being a mono- or a disaccharide, or one of their derivatives or analogues, and:
either W is —OH or —O-G_{S}, Y is H and Z is H;
or W is —OH or —O-G_{S}, Y is —OH or —O-G_{S} and Z is H;
or W is —OH or —O-G_{S}, Y is —OH or —O-G_{S} and Z is —OH or —O-G_{S};
or W is —OH or —OG_{S}, Y is H and Z is —OH or —O-G_{S},
or W is H, Y is —OH or —O-G_{S} and Z is —OH or —O-G_{S};
or W is —OH or —O-G_{S}, Y is alkoxy and Z is H.

9. The method of claim 11 wherein the warm-blooded animals are people seeking to lose weight.

10. The method of claim 11 wherein the warm-blooded animals are people seeking to gain weight.

11. A method of promoting muscle mass to the detriment of fatty mass in warm-blooded animals comprising administrating to warm-blooded in need thereof an effective amount of a Ginkgo extract comprising at least one member or the group consisting of flavoneglycosides, ginkgolide and ginkgolides to treat weight problems.
